(19) United States
(12) Patent Application Publication
(10) Pub. No.: US 2010/0094381 A1
(43) Pub. Date: Apr. 15, 2010

(54) APPARATUS FOR DRIVING ARTIFICIAL RETINA USING MEDIUM-RANGE WIRELESS POWER TRANSMISSION TECHNIQUE

(75) Inventors: Yong Hae KIM, Daejeon (KR); Seung Youl KANG, Daejeon (KR); Byoung Gon YU, Daejeon (KR)

Correspondence Address: AMPACC Law Group, 3500 188th Street S.W., Suite 103, Lynnwood, WA, 98037, US

(73) Assignee: Electronics and Telecommunications Research Institute, Daejeon (KR)

(21) Appl. No.: 12/477,908

(22) Filed: Jun. 4, 2009

(30) Foreign Application Priority Data
Oct. 13, 2008 (KR) 10-2008-0100337

(51) Int. Cl. A61F 9/08; A61F 2/14; A61N 1/36

(52) U.S. Cl. 607/54; 623/6.63

(57) ABSTRACT

Provided is an apparatus for driving an artificial retina using a medium-range power transmission technique. The apparatus can wirelessly transmit power to an artificial retina circuit within a medium range of about 1 m using resonance between a first coil equipped around a user's waist and a second coil implanted in a user's eye. Thus, it is possible to solve the difficulty of implanting a coil in a lens, provide convenience to a user by eliminating the necessity of artificial glasses, and stably supply power to the artificial retina circuit. In addition, it is possible to remarkably lessen the difficulty in connecting the second coil with the artificial retina circuit in an eye.

### CROSS-REFERENCE TO RELATED APPLICATION

This application claims priority to and the benefit of Korean Patent Application No. 10-2008-0100337, filed Oct. 13, 2008, the disclosure of which is incorporated herein by reference in its entirety.

### BACKGROUND

1. Field of the Invention

The present invention relates to an apparatus for driving an artificial retina using a medium-range wireless power transmission technique, and more particularly, to an apparatus for driving an artificial retina which can wirelessly transmit power to an artificial retina circuit within a medium range of about 1 m using resonance between a first coil equipped around a user's waist and a second coil implanted in a user's eye.

2. Discussion of Related Art

An artificial retina is designed for patients for whom a photoreceptor layer of a retina for converting light into an electrical signal is damaged. The artificial retina applies an appropriate electrical signal to optic nerves around the retina, thereby restoring the patient's sight.

Since the artificial retina is implanted in an eye, power cannot be supplied to the artificial retina by a conventional wired connection method. Thus, methods for wirelessly supplying power to the artificial retina are being researched.

FIG. 1 illustrates a conventional method of wirelessly supplying power to an artificial retina.

Referring to FIG. 1, a first coil 111 is equipped in artificial glasses 110, and a second coil 131 is implanted in a lens L of an eye. When power is supplied to the first coil 111 through the artificial glasses 110 from outside, it is transmitted to the second coil 131 by magnetic induction between the first coil 111 and the second coil 131. Thus, the power is supplied to a conversion circuit 140 and an artificial retina circuit 150 through an electric wire 133.

In the wireless power supply method using such magnetic induction, a distance between the first coil 111 and the second coil 131 must be very short, that is, about 1 mm, to enable wireless power transmission. Thus, the second coil 131 must be implanted in the lens L to reduce the distance between the first coil 111 and the second coil 131 as much as possible.

However, the thickness of the lens L is only 4 mm, and thus it is very difficult to implant the second coil 131 in the lens L.

In addition, in the wireless power supply method using magnetic induction, a user must wear the artificial glasses 110. Here, when the artificial glasses 110 slide down and is not in alignment with the lens L, power transmission efficiency suddenly deteriorates, and power supply becomes unstable.

Furthermore, the long electric wire 133 must be connected from the second coil 131 to the artificial retina circuit 150 at the rear of the eye. However, it is very difficult and is not preferable in terms of safety to connect the second coil 131 with the artificial retina circuit 150 through the electric wire 133 in the eye.

### SUMMARY OF THE INVENTION

The present invention is directed to an apparatus for driving an artificial retina capable of wirelessly transmitting power to an artificial retina circuit within a medium range of about 1 m.

More specifically, the present invention is directed to solving the difficulties of implanting a coil in a lens, inconvenience of a user using artificial glasses, unstable power supply due to problems of alignment and distance between the artificial glasses and the lens, and connecting the coil with an artificial retina circuit in the eye.

One aspect of the present invention provides an apparatus for driving an artificial retina using a medium-range wireless power transmission technique, the apparatus wirelessly supplying power to an artificial retina circuit in a user's eye using resonance between a first driver circuit equipped on a specific part of the user's body and a second driver circuit implanted in the eye.

The first driver circuit may include a first coil, a power coil disposed adjacent to the first coil, and a power supply for supplying the power to the power coil, and the second driver circuit may include a second coil having the same resonant frequency as the first coil and a load coil disposed adjacent to the second coil and supplying the power received from the second coil to the artificial retina circuit.

The first coil and the second coil may have helicities in opposite directions, the power coil may have a helicity in the same direction as the helicity of the first coil, and the load coil may have a helicity in the same direction as the helicity of the second coil.

When the power is supplied from the power supply to the power coil in the structure, the power may be transmitted to the first coil by resonance between the power coil and the first coil, and the power transmitted to the first coil may be wirelessly transmitted to the second coil by resonance between the first coil and the second coil. And, when the power is wirelessly transmitted to the second coil, it may be supplied to the artificial retina circuit by resonance between the second coil and the load coil.

The first coil may be equipped on a belt in a winding form. The first coil may have a diameter of 20 to 60 cm, and the second coil may have a diameter of 5 cm or less.

The first coil may have a larger pitch than the second coil such that the first coil and the second coil have the same resonant frequency. The number of turns of the first coil may be larger than the number of turns of the second coil such that the first coil and the second coil have the same resonant frequency.

### BRIEF DESCRIPTION OF THE DRAWINGS

The above and other objects, features and advantages of the present invention will become more apparent to those of ordinary skill in the art by describing in detail exemplary embodiments thereof with reference to the attached drawings, in which:

FIG. 1 illustrates a conventional method of wirelessly supplying power to an artificial retina;

FIG. 2 illustrates an apparatus for driving an artificial retina according to an exemplary embodiment of the present invention;

FIG. 3 is a graph showing power transmission efficiency according to the helicities of a power coil and a load coil shown in FIG. 2;

FIG. 4A is a graph showing power transmission efficiency according to an angle between rotation axes of a first coil and a second coil when the two coils have helicities in the same direction in FIG. 2, and FIG. 4B is a graph showing power transmission efficiency according to an angle between rotation axes of the first coil and the second coil when the two coils have helicities in opposite directions in FIG. 2;

FIG. 5 is a graph showing resonant frequency according to coil diameter; and

FIG. 6 is a graph showing a frequency characteristic according to changes in diameter and pitch of the second coil in FIG. 2.

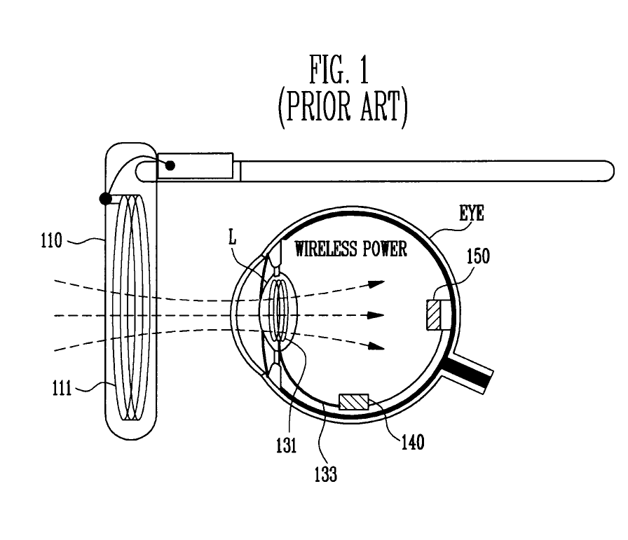

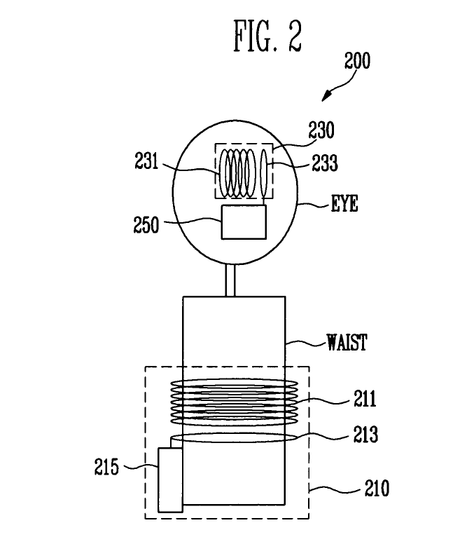

### DETAILED DESCRIPTION OF EXEMPLARY EMBODIMENTS

Hereinafter, exemplary embodiments of the present invention will be described in detail. However, the present invention is not limited to the embodiments disclosed below but can be implemented in various forms. The following embodiments are described in order to enable those of ordinary skill in the art to embody and practice the present invention.

FIG. 2 illustrates an apparatus 200 for driving an artificial retina according to an exemplary embodiment of the present invention.

Referring to FIG. 2, the artificial retina driving apparatus 200 is constituted to wirelessly supply power to an artificial retina circuit 250 in a user's eye using resonance between a first driver circuit 210 equipped on a specific part, e.g., waist, of the user's body and a second driver circuit 230 implanted in the eye.

The first driver circuit 210 includes a first coil 211, a power coil 213 and a power supply 215, and the second driver circuit 230 includes a second coil 231 and a load coil 233.

Preferably, the first coil 211 winds around the user's waist, and more preferably, is equipped on a belt in a winding form for activity.

Here, the first coil 211 may have a diameter of 20 cm to 60 cm, and its number of turns may be 5 to 10.

The helicity of the first coil 211 may be in a clockwise direction or counterclockwise direction. In this exemplary embodiment, the first coil 211 has the helicity in the counterclockwise direction.

The power coil 213 is disposed as close as possible to the first coil 211 but may not be in total contact with the first coil 211 for resonance with the first coil 211.

Only one turn of the power coil 213 is sufficient, and the power coil 213 has a helicity in the same direction as that of the first coil 211. Since the power coil 213 is a single-turn coil, the helicity is determined with respect to a direction from a signal port to a ground port.

In other words, when power is supplied from the power supply 215 to the power coil 213, it is transmitted to the first coil 211 by resonance between the power coil 213 and the first coil 211.

The second coil 231 is implanted in an optic nerve portion at the rear of the eye, and has a helicity in the opposite direction to that of the first coil 211. In this exemplary embodiment, the second coil 231 has a helicity in the clockwise direction.

The load coil 233 is disposed as close as possible to the second coil 231 but may not be in total contact with the second coil 231 for resonance with the second coil 231.

Only one turn of the load coil 233 is sufficient, and the load coil 233 has a helicity in the same direction as that of the second coil 231.

In other words, the power transmitted to the first coil 211 is wirelessly transmitted to the second coil 231 by resonance between the first coil 211 and the second coil 231, and it is supplied to the artificial retina circuit 250 through the load coil 233 by resonance between the second coil 231 and the load coil 233.

The artificial retina circuit 250 includes a rectifier circuit, a photoreceptor circuit, a retinal implant circuit, and so on. The structure of the artificial retina circuit 250 is well known to those of ordinary skill in the art, and thus its detailed description will be omitted.

The artificial retina driving apparatus 200 according to an exemplary embodiment of the present invention has the most remarkable feature of wirelessly transmitting power to the artificial retina circuit 250 within a medium range of about 1 m using resonance between the first coil 211 equipped around a user's waist and the second coil 231 implanted in the user's eye. The medium-range wireless power transmission technique according to an exemplary embodiment of the present invention will be described below in further detail.

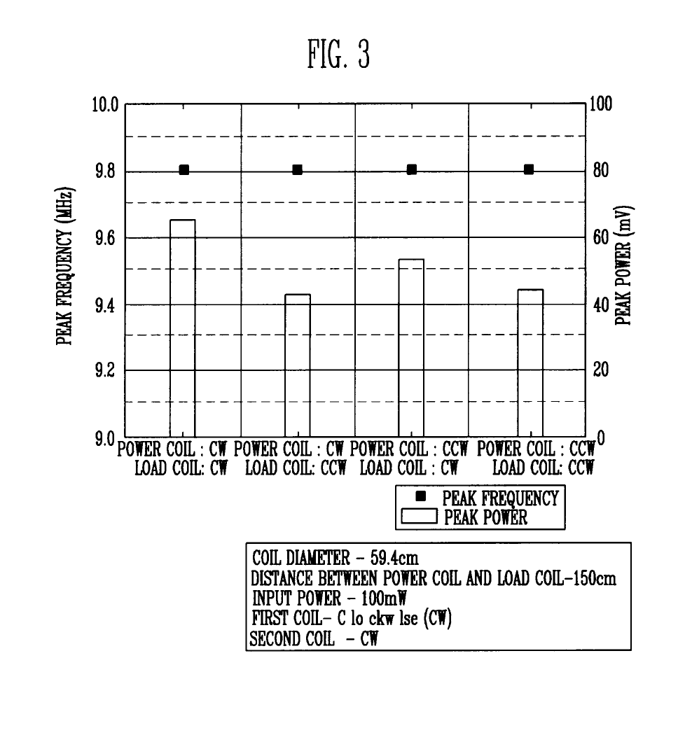

FIG. 3 is a graph showing power transmission efficiency according to the helicities of the power coil 213 and the load coil 233 shown in FIG. 2.

As illustrated in FIG. 3, when the power coil 213 has a helicity in the same direction as the first coil 211 and the load coil 233 has a helicity in the same direction as the second coil 231, the largest power transmission efficiency is obtained.

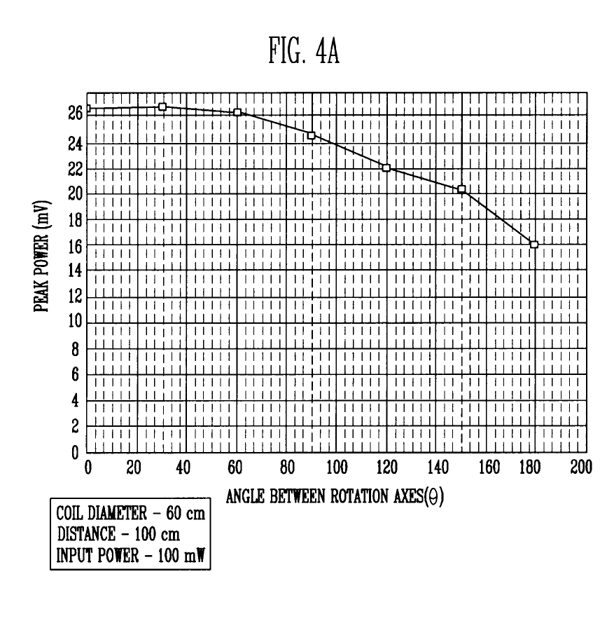

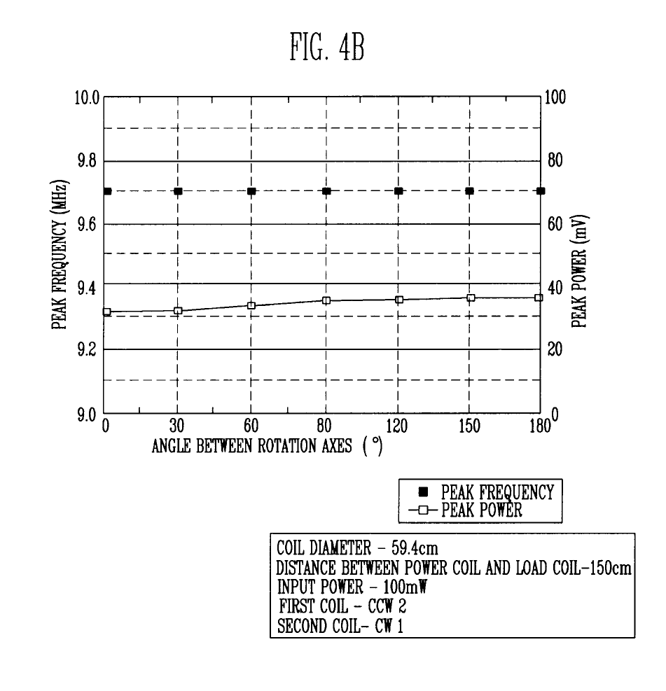

FIG. 4A is a graph showing power transmission efficiency according to an angle between rotation axes of the first coil 211 and the second coil 231 when the two coils have helicities in the same direction in FIG. 2. FIG. 4B is a graph showing power transmission efficiency according to an angle between rotation axes of the first coil 211 and the second coil 231 when the two coils have helicities in opposite directions in FIG. 2.

As illustrated in FIG. 4A, when the first coil 211 and the second coil 231 have helicities in the same direction, power transmission efficiency decreases as the angle between rotation axes of the two coils increases. Here, the variation of power transmission efficiency is large.

As illustrated in FIG. 4B, when the first coil 211 and the second coil 231 have helicities in opposite directions, power transmission efficiency increases as the angle between rotation axes of the two coils increases. Here, the variation of power transmission efficiency is small.

In order to obtain the largest power transmission efficiency in an exemplary embodiment of the present invention, the first coil 211 and the second coil 231 have helicities in opposite directions, the power coil 213 and the first coil 211 have helicities in the same direction, and the load coil 233 and the second coil 231 have helicities in the same direction.

Thus, the artificial retina driving apparatus 200 according to an exemplary embodiment of the present invention can wirelessly supply stable power to the artificial retina circuit 250 within a medium range of about 1 m using resonance between the first coil 211 and the second coil 231 even when the first coil 211 does not have the same rotation axis as the second coil 231.

Meanwhile, the second coil 231 is implanted in an eye and thus must have a diameter of 5 cm or less.

In other words, the second coil 231 must have a tenth of the diameter of the first coil 211 and the same resonant frequency.

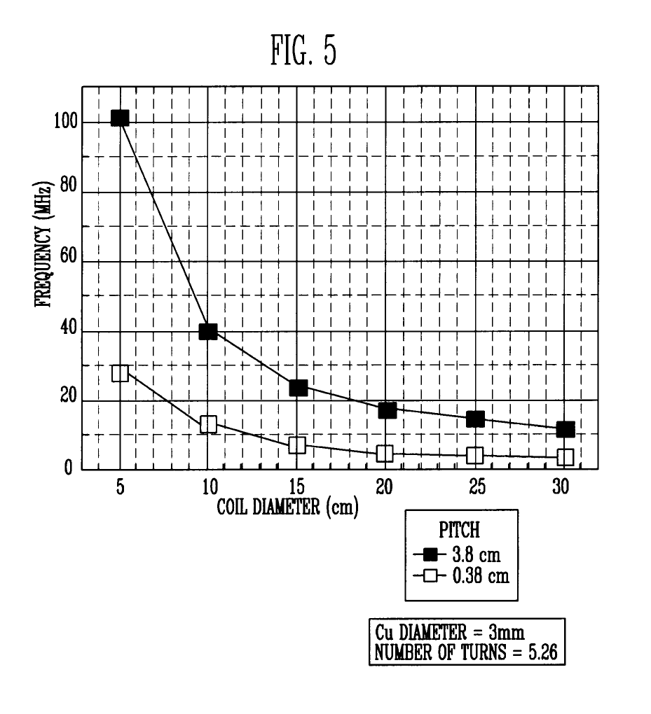

However, when the diameter of a coil decreases, a resonant frequency increases. This can be seen in FIG. 5.

FIG. 5 is a graph showing resonant frequency according to coil diameter. As illustrated in FIG. 5, when the diameter of a coil having a pitch of 3.8 cm is reduced from 10 cm to 5 cm, a resonant frequency increases from 40 MHz to 100 MHz, and when the diameter of a coil having a pitch of 0.38 cm is reduced from 10 cm to 5 cm, a resonant frequency increases from 13 MHz to 28 MHz.

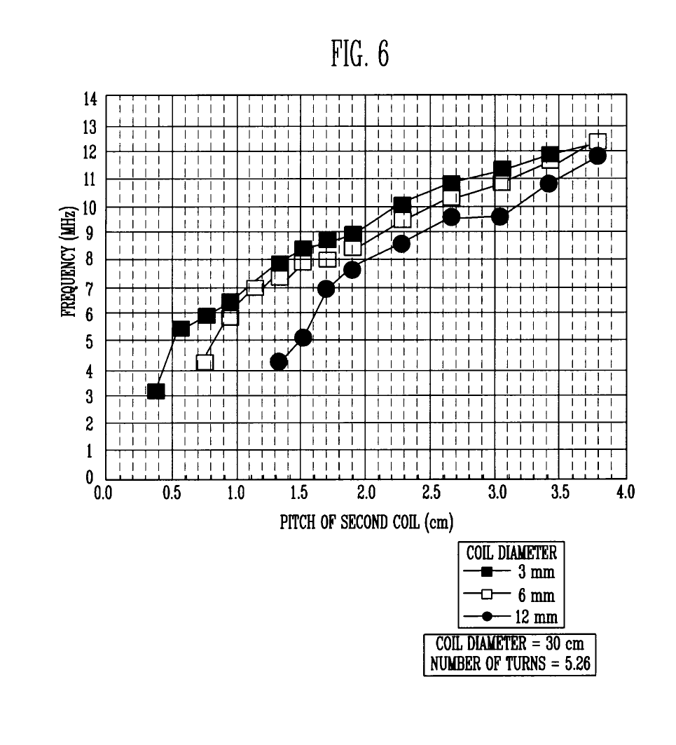

Thus, the second coil 231 having a smaller diameter than the first coil 211 must have a larger pitch than the first coil 211 to have the same resonant frequency as the first coil 211. This will be described in detail below with reference to FIG. 6.

FIG. 6 is a graph showing a frequency characteristic according to changes in diameter and pitch of the second coil 231 in FIG. 2.

As illustrated in FIG. 6, when the diameter of the second coil 231 is reduced and the pitch is increased, power transmission efficiency is improved. Here, the number of turns of the second coil 231 may be made larger than that of the first coil 211 such that the second coil 231 can have the same resonant frequency as the first coil 211.

As described above, when power is supplied from the power supply 215 to the first coil 211 through the power coil 213 with the first coil 211 and the second coil 231 having the same resonant frequency and helicities in opposite directions equipped around a user's waist and implanted in the user's eye, the power is wirelessly transmitted to the second coil 231 by resonance between the first coil 211 and the second coil 231. When the power is wirelessly transmitted to the second coil 231, it is supplied to the artificial retina circuit 250 through the load coil 233 by resonance between the second coil 231 and the load coil 233. As a result, it is possible to wirelessly supply power to the artificial retina circuit 250 within a medium range of about 1 m.

The artificial retina driving apparatus 200 according to an exemplary embodiment of the present invention can solve problems of implanting a coil in a lens, inconvenience of a user using artificial glasses, and unstable power supply due to problems of alignment and distance between conventional artificial glasses and the lens.

In addition, in the artificial retina driving apparatus 200 according to an exemplary embodiment of the present invention, the load coil 233 having a single turn is connected with the artificial retina circuit 250 and implanted in an eye, and then the second coil 231 is disposed adjacent to the load coil 233. Thus, it is possible to remarkably lessen the difficulty in connecting the second coil 231 with the artificial retina circuit 250.

An apparatus for driving an artificial retina according to an exemplary embodiment of the present invention can wirelessly transmit power to an artificial retina circuit within a medium range of about 1 m using resonance between a first coil equipped around a user's waist and a second coil implanted in a user's eye.

Therefore, it is possible to solve the difficulty of implanting a coil in a lens, provide convenience to a user by eliminating the necessity of artificial glasses, and stably supply power to the artificial retina circuit. In addition, it is possible to remarkably lessen the difficulty of connecting the second coil with the artificial retina circuit.

While the invention has been shown and described with reference to certain exemplary embodiments thereof, it will be understood by those skilled in the art that various changes in form and details may be made therein without departing from the spirit and scope of the invention as defined by the appended claims.

What is claimed is:

1. An apparatus for driving an artificial retina using a medium-range wireless power transmission technology, wherein power is wirelessly supplied to an artificial retina circuit in a user's eye by resonance between a first driver circuit equipped on a specific part of the user's body and a second driver circuit implanted in the user's eye.

2. The apparatus of claim 1, wherein the first driver circuit includes a first coil, a power coil disposed adjacent to the first coil, and a power supply for supplying the power to the power coil, and the second driver circuit includes a second coil having the same resonant frequency as the first coil and a load coil disposed adjacent to the second coil and supplying the power received from the second coil to the artificial retina circuit.

3. The apparatus of claim 2, wherein the first coil and the second coil have helicities in opposite directions.

4. The apparatus of claim 3, wherein the power coil has a helicity in the same direction as the helicity of the first coil.

5. The apparatus of claim 3, wherein the load coil has a helicity in the same direction as the helicity of the second coil.

6. The apparatus of claim 2, wherein when the power is supplied from the power supply to the power coil, the power is transmitted to the first coil by resonance between the power coil and the first coil, and the power transmitted to the first coil is wirelessly transmitted to the second coil by resonance between the first coil and the second coil.

7. The apparatus of claim 6, wherein, when the power is wirelessly transmitted to the second coil, the power is supplied to the artificial retina circuit by resonance between the second coil and the load coil.

8. The apparatus of claim 2, wherein the first coil is equipped on a belt in a winding form.

9. The apparatus of claim 2, wherein the first coil has a diameter of 20 cm to 60 cm, and the second coil has a diameter of 5 cm or less.

10. The apparatus of claim 9, wherein the first coil has a larger pitch than the second coil such that the first coil and the second coil have the same resonant frequency.

11. The apparatus of claim 9, wherein the number of turns of the first coil is larger than the number of turns of the second coil such that the first coil and the second coil have the same resonant frequency.
